(19) United States
(12) Patent Application Publication
(10) Pub. No.: US 2004/0101881 A1
(43) Pub. Date: May 27, 2004

(54) Surfactant/oxidizing agent solution and methods of use

(76) Inventors: Gerard Durmowicz, Timonium, MD (US); Christine Martinaitis, Columbia, MD (US); Tiffiny Poth, Westminster, MD (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/470,762

(22) PCT Filed: Feb. 1, 2002
(86) PCT No.: PCT/US02/02719

(51) Int. Cl. C12Q 1/68; C12P 19/34

(52) U.S. Cl. 435/6; 435/91.2; 510/161

(57) ABSTRACT

A solution is disclosed which is useful in removing contaminating nucleic acids from various surfaces, including areas such as laboratory bench tops, floors and walls, as well as surfaces of instruments and equipment in laboratories where nucleic acid amplification reactions are conducted. Further, methods and kits are provided that afford effective decontamination and removal of nucleic acids from such surfaces.

### BACKGROUND OF THE INVENTION

Solutions comprising oxidizing agents, such as hypohalous acids, such as sodium hypochlorite, or bleach, render nucleic acid molecules unamplifiable by known nucleic acid amplification methods such as Strand Displacement Amplification (SDA), Polymerase Chain Reaction (PCR) (U.S. Pat. Nos. 4,683,195 and 4,683,202), Ligase Chain Reaction (LCR) (EP 0320308) Transcription Mediated Amplification (TMA), 3SR, Nucleic Acid Sequence Based Amplification (NASBA) (U.S. Pat. No. 5,409,818), Rolling Circle Amplification (U.S. Pat. No. 6,280,949) and others. Contact of the sodium hypochlorite solution, for example, with a nucleic acid molecule chlorinates the nucleic acid molecule causing permanent destabilization of the double helix structure. The resulting chemically modified molecule no longer can be replicated by enzymatic means, and thus, the nucleic acid molecule is rendered unamplifiable (U.S. Pat. No. 5,612,200).

In general, such hypohalous acids destabilize nucleic acids by halide transfer to amino groups on DNA causing oxidative damage (Shishido et al., Redox Rep (2000) 5(4):243-7). Hence, for example, sodium hypochlorite solutions are commonly used to wash and rinse surfaces areas where nucleic acid amplification reactions are conducted. Such washes and rinses can and are referred to as nucleic acid decontamination procedures. Commonly, such surfaces include laboratory bench tops, floors and walls, as well as surfaces of instruments and equipment in laboratories where such nucleic acid amplification reactions are conducted.

However, it is also known that, oftentimes, such sodium hypochlorite washes and rinses do not render all nucleic acid molecules unamplifiable. Furthermore, the washing and rinsing with sodium hypochlorite solution do not remove either amplifiable or unamplifiable nucleic acid molecules from the treated surface.

Thus, there is recognized in the art, a need for a more effective nucleic acid decontamination and removal composition and method.

### SUMMARY OF THE INVENTION

To address that recognized need in the art, the present invention comprises a solution of a nucleic acid oxidizing agent and a surfactant. It has been found that the use of this solution is more effective to provide nucleic acid decontamination and removal than conventional sodium hypochlorite solutions.

In one aspect a solution for removing and helix-destabilizing nucleic acids on surfaces is envisaged comprising a blend of surfactant for suspending and a nucleic acid oxidizing agent for destabilizing nucleic acids by oxidation, rendering such unamplifiable. Surfaces treated with such a solution for an appropriate period of time have greater than about 50% of the amplification detectable nucleic acids destabilized and removed. More preferably, at least 90% of the amplification detectable nucleic acids destabilized and removed.

In another aspect, the surfactant may comprise higher fatty acid alkali soaps and organic builder salts such that anionic, non-ionic, ampholytic or zwitterionic detergents are formed. In a related aspect, such detergents may comprise sodium alkylaryl sulfonate, alcohol sulfate, phosphate and carbonates. Oxidizing agents include, but are not limited to, HOBr, HOI, HOCI and other hypohalous acids. Other oxidizing agents include, but are not limited to, peroxides, such as H₂O₂ and perioxynitrite.

A preferred oxidizing agent is a hypochlorite. Hypochlorites include, but are not limited to sodium, lithium, calcium and dibasic magnesium hypochlorite.

The ratio of surfactant to oxidizing agent can be varied such that a sufficient oxidizing agent availability is maintained to cause at least 50% of contaminating nucleic acids to become oxidized and thus rendered unamplifiable. Preferably greater than 50% of nucleic acids are rendered unamplifiable and removed. It is preferred that more than 60%, 70%, 80% or more than 90% of nucleic acids are rendered unamplifiable and removed.

In another aspect, the solution is exposed to surfaces for such a time as to remove detectable nucleic acids and oxidize sufficient nucleic acid molecules, resulting in a decontaminated surface.

In another aspect, the invention envisages a kit comprising surfactant and oxidizing agent as separate components. These separate components are to be mixed just prior to contacting suspect surfaces such that the resulting solution may decontaminate these surfaces, where contaminating nucleic acids are rendered suspensable and unamplifiable by oxidation (e.g., chlorination) and nucleic acids are removed by contact with admixed components.

Methods of decontaminating surfaces comprising contacting such surfaces with a solution are also envisaged. In a related aspect, a surface suspected of being contaminated with nucleic acids is treated for a time with the solution to suspend, oxidize and destabilize undesired nucleic acids. Such methods include contacting surfaces for at least 30 seconds and as long as until the composition of interest dries on the surface. The solution of interest finds use at normal, ambient temperature, such as between about 20° C. to about 40° C. but can be used at colder or warmer temperatures. The treated surface is followed with an aqueous rinse (e.g., but not limited to, deionized water) to rehydrate and to remove the solution and nucleic acids, which is then followed by wiping and rinsing of said contacted surface such that at least 50% of the contaminating nucleic acids are oxidized and removed, and preferably greater than 90% of contaminating nucleic acids are oxidized and removed from the surface.

These and other advantages associated with the present invention and a more detailed explanation of preferred embodiments is described below.

### DETAILED DESCRIPTION OF THE INVENTION

The term “unamplifiable” and grammatical variations thereof, herein means that a nucleic acid can no longer be replicated by enzymatic means.

The term “solution” or “non-particulate solution” and grammatical variations thereof, herein means an essentially single-phase liquid system. However, as well known in the art, at saturating levels of surfactant there may be particulate precipitation of said surfactant. Under these circumstances, such a saturated solution and particularly the fluid phase is considered a non-particulate solution for the purposes of the instant invention.

The term “surfactant” or “surface acting agent” herein means that any compound that reduces surface tension when dissolved or suspended in water or water solutions, or which reduces interfacial tension between two liquids, or between a liquid and a solid. In a related aspect, there are at least three categories of surface active agents: detergents, wetting agents, and emulsifiers; all use the same basic chemical mechanism and differ, for example, in the nature of the surfaces involved.

The term “nucleic acid halogenating” or oxidizing agent” and grammatical variations thereof, herein means an element, compound, composition and the like that halogenates or oxidizes nucleic acids, wherein such halogenation or oxidation prevents complementary base pairing of the strands and alters the structure of the nucleic acid such that amplification of single-stranded and double-stranded nucleic acids cannot occur.

The term “detergent” and grammatical variations thereof, herein means an emulsifying agent or surface active agent made usually by action of alkali on fat or fatty acids and consisting essentially of sodium or potassium salts of such acids. In a related embodiment, the term may include any of numerous synthetic water-soluble or liquid organic preparations that are chemically different from soaps but are able to emulsify oils, hold dirt in suspension, and act as wetting agents.

The term “helix destabilizing” and grammatical variations thereof, herein means an effect on the double helix of nucleic acids such that complementary base pairing cannot be maintained between nucleic acid strands treated with the surfactant/oxidizing agent solution.

The term “amplification detectable” and grammatical variations thereof, herein means nucleic acid present at a concentration such that detection is capable following nucleic acid amplification.

Amounts of solid reagents are expressed as w/v, for example, grams per milliliter or grams per liter. Amounts of liquid reagents are expressed as v/v, for example, milliliters per liter.

The invention relates to a new composition of matter and nucleic acid decontamination procedure using such composition of matter. The composition of matter is a combination of a surfactant (e.g., detergent) and a nucleic acid oxidizing agent (e.g., hypohalous acid). Although virtually any surfactant is useful, anionic detergents are preferred. For example, one such anionic detergent is a proprietary blend of sodium linear alkylaryl sulfonate, alcohol sulfate, phosphates and carbonates known as ALCONOX®. ALCONOX® is available as a powdered detergent. A similar detergent, LIQUINOX®, also is useful in the present invention.

In a preferred embodiment, the oxidizing agent is hypochlorite and in a more preferred embodiment, the hypochlorite is sodium hypochlorite.

The concentration of surfactant is between about 0.1% and saturation. A preferred amount is from about 0.2% to about 10%. A suitable amount is from about 0.5% to about 5%. A preferred range is from about 0.6% to about 1%. When ALCONOX® is used as the surfactant, the final concentration of ALCONOX® is preferably about 0.75% w/v.

The concentration of oxidizing agent is between about 0.01% and 10%. A preferred range is from about 0.05% to about 5%. A suitable amount is from about 0.1% to about 3%. In the case where the oxidizing agent is sodium hypochlorite, the hypochlorite concentration is between about 0.01% and about 5%, preferably about 0.05% to about 3%, and preferably between about 0.1% to about 2%. In a related embodiment, the final concentration of hypochlorite is 1%.

Water-soluble salts of the higher fatty acids, i.e., “soaps,” are useful surfactants in the blends of solution disclosed herein (see also U.S. Pat. No. 4,123,377). This class of surfactants includes ordinary alkali metal soaps such as the sodium, potassium, ammonium and alkanol ammonium salts of higher fatty acids containing from about 8 to about 24 carbon atoms and preferably from about 10 to about 20 carbon atoms. Soaps can be made by direct saponification of fats and oils or by the neutralization of free fatty acids. Particularly useful are the sodium and potassium salts of the mixtures of fatty acids derived from coconut oil and tallow, i.e., sodium or potassium tallow and coconut soaps.

Another class of anionic surfactants includes water-soluble salts, particularly the alkali metal, ammonium and alkanolammonium salts, of organic sulfuric reaction products having in their molecular structure an alkyl group containing from about 8 to about 22 carbon atoms and a sulfonic acid or sulfuric acid ester group. (Included in the term “alkyl” is the alkyl portion of acyl groups.) Examples of this group of synthetic surfactants which can be used in the present solutions are the sodium and potassium alkyl sulfates, especially those obtained by sulfating the higher alcohols (C₈-C₁₈ carbon atoms) produced by reducing the glycerides of tallow or coconut oil, sodium and potassium C₈-C₂₀ paraffin sulfonates, and sodium and potassium alkyl benzene sulfonates, in which the alkyl group contains from about 9 to about 15 carbon atoms in straight chain or branched chain configuration, e.g., those of the type described in U.S. Pat. No. 2,220,099, and 2,477,383, incorporated herein by reference.

Other anionic surfactant compounds useful herein include the sodium alkyl glyceryl ether sulfonates, especially those ethers or higher alcohols derived from tallow and coconut oil, sodium coconut oil fatty acid monoglyceride sulfonates and sulfates; and sodium or potassium salts of alkyl phenol ethylene oxide ether sulfate containing about 1 to about 10 units of ethylene oxide per molecule and wherein the alkyl groups contain about 8 to about 12 atoms.

Other useful anionic surfactants herein include the water-soluble salts of esters of α-sulfonated fatty acids containing from about 6 to 20 carbon atoms in the ester group; water-soluble salts of 2-acyloxy-alkane-1-sulfonic acids containing from about 2 to 9 carbon atoms in the alkane moiety; alkyl ether sulfates containing from about 10 to 20 carbon atoms in the alkyl group and from about 1 to 30 moles of ethylene oxide; water-soluble salts of olefin sulfonates containing from about 10 to 20 carbon atoms in the alkyl group and from about 1 to 30 moles of ethylene oxide; water-soluble salts of olefin sulfonates containing from about 12 to 24 carbon atoms; and β-alkoxy alkane sulfonates containing from about 1 to 3 carbon atoms in the alkyl group and from about 8 to 20 carbon atoms in the alkane moiety.

Preferred water-soluble anionic organic surfactants herein include linear alkyl benzene sulfonates containing from about 11 to 14 carbon atoms in the alkyl group; the tallow range alkyl sulfates; the coconut range alkyl glyceryl sulfonates; and alkyl ether sulfates wherein the alkyl moiety contains from about 14 to 18 carbon atoms and wherein the average degree of ethoxylation varies between 1 and 6.

Specific preferred anionic surfactants for use herein include: sodium linear C₁₀-C₁₂ alkyl benzene sulfonate; triethanolamine C₁₀-C₁₂ alkyl benzene sulfonate; sodium tallow alkyl sulfate; sodium coconut alkyl glyceryl ether sulfonate; and the sodium salt of a sulfated condensation product of tallow alcohol with from about 3 to about 10 moles of ethylene oxide.

It is to be recognized that any of the foregoing anionic surfactants can be used separately herein or as mixtures. Mixtures of anionic surfactants which can be used in the present detergent compositions can be taken from the sodium and potassium alkyl sulfates, especially those obtained by sulfating the higher alcohols (C₈-C₁₈ carbon atoms) produced by reducing the glycerides of tallow or coconut oil, sodium and potassium C₈-C₂₀ paraffin sulfonates, sodium and potassium alkyl benzene sulfonates, in which the alkyl group contains from about 9 to about 15 carbon atoms in straight chain or branched chain configuration, and the sodium salt of a sulfated condensation product of tallow alcohol with from about 2 to about 10 moles of ethylene oxide. Nonionic surfactants include the water-soluble ethoxylates of C₁₀-C₂₀ aliphatic alcohols and C₆-C₁₂ alkyl phenols.

Semipolar surfactants useful herein include water-soluble amine oxides containing one alkyl moiety of from about 10 to 28 carbon atoms and 2 moieties selected from the group consisting from 1 to about 3 carbon atoms; water-soluble phosphine oxides containing one alkyl moiety of about 10 to 28 carbon atoms and 2 moieties selected from the group consisting of alkyl groups and hydroxyalkyl groups containing from about 1 to 3 carbon atoms; and water-soluble sulfoxides containing one alkyl moiety of from about 10 to 28 carbon atoms and a moiety selected from the group consisting of alkyl and hydroxyalkyl moieties of from 1 to 3 carbon atoms.

Ampholytic surfactants include derivatives of aliphatic or aliphatic derivatives of heterocyclic secondary and tertiary amines in which the aliphatic moiety can be straight chain or branched and wherein one of the aliphatic substituents contains from about 8 to 18 carbon atoms and at least one aliphatic substituent contains an anionic water-solubilizing group.

Zwitterionic surfactants include derivatives of aliphatic quaternary ammonium, phosphonium and sulfonium compounds in which the aliphatic moieties can be straight or branched chain, and wherein one of the aliphatic substituents contains from about 8 to 18 carbon atoms and one contains an anionic water-solubilizing group.

It is further envisaged to use common laboratory surfactants and detergents to practice the instant invention such as, but not limited to, the Tween series, the octylphenol series (Triton), tergitol detergents (NP series), sodium laureth sulfide (SDS), Brij detergents and niaproff anionic detergents. Useful builders herein include any of the conventional inorganic and organic water-soluble builder salts, as well as various water-insoluble and so-called “seeded” builders.

Inorganic detergency builders useful herein include, for example, water-soluble salts of phosphates, pyrophosphates, orthophosphates, polyphosphates, phosphonates, carbonates, bicarbonates, borates and silicates. Specific examples of inorganic phosphate builders include sodium and potassium tripolyphosphates, phosphates, and hexametaphosphates. The polyphosphonates specifically include, for example, the sodium and potassium salts of ethylene diphosphonic acid, the sodium and potassium salts of ethane 1-hydroxy-1, 1-diphosphonic acid, and the sodium and potassium salts of ethane-1,1,2-triphosphonic acid. Examples of these and other phosphorous builder compounds are disclosed in U.S. Pat. Nos. 3,159,581; 3,213,030; 3,422,021; 3,422,137; 3,400,176; 3,400,148; 4,019,998 and 4,019,999, incorporated herein by reference. Sodium tripolyphosphate is an especially preferred, water-soluble inorganic builder herein.

Non-phosphorous containing sequestrants can also be selected for use herein as detergency builders. Specific examples of non-phosphorus, inorganic builder ingredients include water-soluble inorganic carbonate, bicarbonate, borate and silicate salts. The alkali metal, e.g., sodium and potassium, carbonates, bicarbonates, borates (Borax) and silicates are particularly useful herein.

Water-soluble, organic builders are also useful herein. For example, the alkali metal, ammonium and substituted ammonium polyacetates, carboxylates, polycarbonates, succinates, and polyhydroxysulfonates are useful builders in the present compositions and processes. Specific examples of the polyacetate and polycarboxylate builder salts include sodium potassium, lithium, ammonium and substituted ammonium salts of ethylene diamine tetraacetic acid, nitrilotriacetic acid, oxydisuccinic acid, mellitic acid, benzene polycarboxylic acids and citric acid.

Other preferred non-phosphorous builder materials (both organic and inorganic) herein include sodium carbonate, sodium bicarbonate, sodium silicate, sodium citrate, sodium oxydisuccinate, sodium mellitate, sodium nitrilotriacetate, and sodium ethylenediaminetetraacetate, the mixtures thereof.

Preferably, the blend is solvated in an aqueous solution, however, other solvents are contemplated as useful for the present invention. For example alcohols, such as ethanol, methanol, propanol, isopropyl alcohol, butanol and the like can be used. Such non-water solvents can serve as the sole solvent, or if miscible, can be combined with water. The composition of interest can include a buffer to facilitate solvation and to yield a solution of reduced acidity or alkalinity.

The composition of interest also contains a nucleic acid oxidizing agent agent. The agent oxidizes nucleic acids and thereby prevents proper complementary base pairing. The agent also alters the nucleic acid so that amplification thereof is not possible. Suitable oxidizing agents include, but are not limited to, hypohalous acids. Suitable hypohalous acids include HOBr, HOI and HOCI. Other agents include, but are not limited to, peroxides, such as H₂O₂ and perioxynitrite.

A preferred oxidizing agent is hypochlorite.

Hypochlorites (bleach) useful in the blend of the present invention include sodium, lithium, calcium and di-basic magnesium. Sodium hypochlorite is preferred.

Certain oxidizing agents yield alkaline solutions, as do many surfactants. Thus, for example, hypohalous acids, such as sodium hypochlorite, yield solutions with pH values well greater than 7. However, higher pH values also can impact nucleic acid stability and thus, can be beneficial.

In another embodiment, the combination of surfactant and oxidizing agent is such that inactivation and the removal of greater than 50% of the contaminating nucleic acids occurs in at least about 30 seconds. In a related embodiment, the contact time is sufficient to cause more than 90% of the contaminating nucleic acid to be rendered unamplifiable and removed.

In a related aspect, when the surface is in contact with the solution for at least about 2 minutes, greater than about 50% of contaminating amplification detectable nucleic acids are removed. Further, 2 minutes contact time is sufficient to cause about 95% of the contaminating amplification detectable nucleic acids to be rendered unamplifiable and removed.

The use of a surfactant/oxidizing agent solution allows virtually no redeposition of removed (and unwanted) nucleic acid molecules. More specifically, it is believed that when a surfactant is used during decontamination procedures, nucleic acid molecules, including target molecules as well as amplicons are lifted from a surface, solubilized and are not redeposited. Contact of the oxidizing agent solution with a nucleic acid molecule oxidizes the nucleic acid molecule causing permanent destabilization of the double helix structure and rendering nucleic acids unamplifiable. Solubilized nucleic acids are more accessible to the oxidizing agent. The resulting chemically modified molecule can no longer be replicated by enzymatic means and thus the nucleic acid molecule is rendered unamplifiable. The surfactant/oxidizing agent solutions of the present invention not only render nucleic acid molecules unamplifiable, but also easily removable from surfaces.

Use of the surfactant/oxidizing agent solutions of the present invention involves the application of such solutions to surfaces. Preferably the surfaces are soaked with the solution. The solution is left on the surface for a period of time, for example, at least about 30 seconds, preferably for about one minute and more preferably more than about two minutes up through the time of having the solution of interest dry on the surface. Then the surface is wiped with an aqueous solution, such as water, water/alcohol mixture and the like, which optionally can be buffered, to remove the nucleic acid molecules. The surfaces should be rinsed with water or like solvent and wiped to remove any residual surfactant/oxidizing solution and nucleic acid molecules remaining.

The solution can be contacted with a surface at room temperature that generally is in the range of 220-28° C., and more generally 24-26° C. However, the solution is effective at temperatures varying from 0° C. to about 97-99° C. The optimal temperature is adjustable depending on the surfactant and oxidizing agent used. Suitable additives, such as buffers and salts, can be added to ensure use of the solution at warmer or colder temperatures. In a related embodiment, the preferred temperature for contamination removal is between about 10° C. and about 40° C.

Sodium hypochlorite alone has limitations in being only about 50% effective in removing DNA. Experimental evidence shows that application of a conventional sodium hypochlorite solution to a nucleic acid amplification instrument contaminated with target nucleic acid molecules did not fully remove the nucleic acid molecules causing such contamination. However, the use of a detergent (ALCONOX®)/sodium hypochlorite solution in accordance with the present invention eliminated the nucleic acid molecules causing contamination to a greater degree. The detergent/bleach solution was applied to the same instrument after use of bleach alone for comparison. Similarly, environmental data has been generated from laboratories where nucleic acid amplification reactions are conducted showing that use of a surfactant/oxidizing agent solution in accordance with the present invention results in decreased nucleic acid contamination as compared to the application of a conventional sodium hypochlorite solution.

It is preferred that the solutions be prepared shortly before use. Thus, various types of pre-packaging of an appropriate amount of surfactant and oxidizing agent are envisioned. Such pre-packaging may facilitate dissolving of the surfactant, oxidizing agent or both into solution, and may comprise a vehicle for delivery of the components into the solution, such as a dissolvable vehicle. The present invention also comprises a kit containing appropriate amounts of surfactant and oxidizing agent in separate containers for mixing prior to application.

In one embodiment, a kit comprising a separate container consists essentially of a surfactant and a separate container consisting essentially of oxidizing agent in concentrations such that when admixed, a non-particulate solution for removing and helix destabilizing contaminating nucleic acids on amplification reaction surfaces is generated. In a related embodiment, when the surfaces are contacted with the decontaminating solution the undesired nucleic acids are rendered unamplifiable by oxidation and greater than about 50% of amplification detectable nucleic acids are removed.

In another embodiment, the kit comprising a separate container consisting essentially of a higher fatty acid alkali metal soap, organic builder salts and a separate container consisting essentially of oxidizing agent.

The following non-limiting examples illustrate the efficacy and advantages associated with the solution for certain surfaces in accordance with the present invention. It is understood that these examples are for illustration purposes only and that alternative embodiments are contemplated as within the scope of the present invention.

### EXAMPLES

### Example 1

### Effectiveness of Bleach/ALCONOX Solution in Removing DNA Environmental Contamination

A preferred protocol involves taking a set of pre-cleaning swabs, cleaning the area with 1% sodium hypoochlorite/0.75% ALCONOX® detergent solution, and taking a set of post cleaning swabs. Swabs generally are processed on the day of collection but can be held and used within 4-6 days of collection if stored at 2-27° C. The swab is placed into a suitable vessel, such as a test tube, containing a diluent, such as a standard nucleic acid amplification buffer solution, and swirled in the diluent for about 5-10 seconds. The swab is expressed on the inside wall of the vessel to remove as much of the liquid containing any nucleic acids collected in the swab into the liquid diluent. The swab is discarded and the tube vortexed. A sample is taken for detection of nucleic acid by an amplification method.

This experiment used a pre-test/post-test design to evaluate the effectiveness of 1% sodium hypochlorite/0.75% ALCONOX® detergent solution as a DNA decontamination agent. In the pre-test, contaminated areas were swabbed 10 times to assess the degree of contamination. These areas were then treated with 1% sodium hypochlorite/0.75% ALCONOX® detergent solution and 10 post-test swabs were collected. Presence of nucleic acid was by the strand displacement method using the BDProbeTec™ ET test kit to detect Chlamydia trachomalis (CT) and Neisseria gonorrhoeae (GC) nucleic acids. The swab diluent provided in the kit was used. A 50% reduction in the frequency of positive contamination monitoring swabs was considered to be an indicator of effective decontamination. A CT/GC score of <2000 is considered negative for the strand separation assay as performed.

Data from the contaminated areas are summarized in TABLE 1, below.

TABLE 1
CT/GC Contaminated Areas, 6 Areas, 20 Swabs from each Area.

| | CT-GC/20 | CT-GC/20 | |
|---|---|---|---|
| Area | Swabs Before | Swabs After | % Reduction |
| 1 | 7/20 | 0/20 | 100% |
| 2 | 8/20 | 0/20 | 100% |
| 3 | 6/20 | 0/20 | 100% |
| 4 | 5/20 | 0/20 | 100% |
| 5 | 9/20 | 0/20 | 100% |
| 6 | 10/20 | 1/20¹ | 90% |
| Overall | 45/120 | 1/120 | 99.60% |

TABLE 2
Breakdown of Data Before Cleaning.

| Area | Before GC | Before CT |
|---|---|---|
| 1 | 7/10 | 0/10 |
| 2 | 8/10 | 0/10 |
| 3 | 6/10 | 0/10 |
| 4 | 2/10 | 3/10 |
| 5 | 9/10 | 0/10 |
| 6 | 10/10 | 0/10 |

Acceptance criterion for this validation called for at least a 50% reduction in the frequency of positives in contamination monitoring samples. Each individual test area as well as the pooled data sets met this criterion.

These data demonstrate that 1% sodium hypochlorite/0.75% ALCONOX® detergent solution is an effective agent for the removal of DNA amplification products form laboratory surfaces and equipment.

### Example 2

### Use of Peroxide as the Oxidizing Agent

Hydrogen peroxide was used in combination with ALCONOX®. Two different concentrations of hydrogen peroxide were used, 1% and 3% in distilled water, with the detergent used at the same concentrations.

TABLE 3*
Performance with Hydrogen Peroxide.

| | | Before | | After | |
|---|---|---|---|---|---|
| Swab # | Condition** | CT | GC | CT | GC |
| 1 | A | 2086 | 7 | 47 | 9 |
| 2 | A | 5181 | 48 | 159 | 98 |
| 3 | B | 0 | 21633 | 663 | 1 |
| 4 | B | 7845 | 21586 | 309 | 148 |
| | Pos. Ctrl. | 33796 | 53673 | 16488 | 36667 |
| | Neg. Ctrl. | 420 | 288 | 378 | 177 |

*Controls were run concurrently with each sample to determine the effects of the particular solutions on amplification reaction.
**Conditions: A, 1% hydrogen peroxide + 1% ALCONOX ^{®}; B, 3% Hydrogen peroxide + 3% ALCONOX ®.

### Example 3

### Performance of Swab Testing for Amplicons before and after Cleaning

An ALCONOX® and bleach solution was used to clean an instrument directed to operate with the SDA amplification assay. The solution was prepared by adding 600 ml of a commercial bleach solution (5% sodium hypochlorite) was added to a 3.5 L container followed by 22.5 g of ALCONOX®. Warm water (qs to 3000 ml) was added to produce the final solution that was used to wipe down the suspected contaminated surfaces of the instrument. The washing procedure was repeated three to four times, the solution and towels were changed after each wash, before the certain areas were swabbed.

Swabs were dipped into swab sample diluent and then swabbed over different locations that were previously shown to be contaminated with amplicons. Bleach, detergent and water were added as in Example 1. The procedure for wiping the surfaces was also as provided in Example 1, except that the procedures were repeated 4 times instead of 3. Amplicon monitoring using dipped swabs was as described in Example 1.

Data from the contaminated areas are summarized in TABLE 4, below.

TABLE 4*
CT/GC Contaminated Areas Before and at Final Cleaning.

| | | Before Cleaning | | After Cleaning | |
|---|---|---|---|---|---|
| Swab # | Location | CT | GC | CT | GC |
| 1 | Matrix Stand | 38 | 17599 | 301 | 31 |
| 2 | Printer-Right | 3018 | 280 | 195 | 106 |
| | Side | | | | |
| 3 | Printer-Top | 8 | 8046 | 38 | 95 |
| 4 | Lysis Rack Top, | 657 | 10675 | 20 | 21 |
| | Handles | | | | |
| 5 | Heat Blocks-Left | 2895 | 13630 | 1 | 140 |
| | Side | | | | |
| 6 | Heat Blocks- | 2012 | 3472 | 21 | 14 |
| | Right Side | | | | |
| 7 | Heat Blocks-Heat | 162 | 7542 | 153 | 86 |
| | Spike | | | | |
| 8 | Heat Blocks-Pre- | 6 | 25095 | 0 | 0 |
| | Warm | | | | |
| 9 | Heat Blocks- | 76 | 18483 | 60 | 7 |
| | Front Surface | | | | |
| 10 | Lysis Block-Top | 36 | 13994 | 13 | 5 |
| 11 | Lysis Block- | 0 | 12142 | 163 | 70 |
| | Sides | | | | |
| 12 | Black Cord | 95 | 28947 | 31 | 29 |
| 13 | Power Cord to | 18 | 10772 | 157 | 65 |
| | Printer | | | | |
| 14 | Printer Cable | 0 | 16266 | 407 | 105 |
| 15 | Black Cord | 1 | 7123 | 563 | 144 |
| 16 | Black Cord | 118 | 12409 | 7 | 46 |
| | Pos. Ctrl. | 20977 | 35914 | 23157 | 18075 |
| | Neg. Ctrl. | 44 | 98 | 559 | 144 |

*Controls were run concurrently with each sample to determine the effects of solution on amplification reaction.

Peripherals from the instrument were found to be contaminated. After thoroughly cleaning each piece of equipment with the solution of interest, no amplicon contamination was found.

### Example 4

### Environmental Swab Testing on an Instrument

Various areas on the instrument and peripherals were swabbed and tested using the CT/GC SDA assay. After results were collected, the instrument and peripherals were cleaned using an ALCONOX@ and bleach solution. The procedure was as in Example 1, except that the procedure was repeated 2×.

Data from the contaminated areas are summarized in TABLE 5, below.

TABLE 5*
CT/GC Contaminated Areas from the Field.

| | | Before Cleaning | | After Cleaning | |
|---|---|---|---|---|---|
| Swab # | Location | CT | GC | CT | GC |
| 1 | Latch | 296 | 2362 | 102 | 0 |
| 2 | Front Deck | 124 | 7899 | 30 | 100 |
| 3 | Right Outside | 134 | 9925 | 60 | 61 |
| 4 | Heat Block-Rt. | 2184 | 159 | 0 | 4 |
| | Side | | | | |

*Controls were run concurrently with each sample to determine the effects of solution on amplification reaction.

After through cleaning with the solution of interest, all tested areas of the equipment were found to be contamination free.

### Example 5

### Additional Testing

An SDA instrument was tested for contamination and decontamination. Fourteen areas were swabbed with a 1% solution hypochlorite. Ten areas were tested after cleaning with a solution of interest as prepared in Example 1.

Of the fourteen sites tested with bleach alone, 7/14 were positive for GC and 0/14 were positive for CT. On the other hand, none of the ten areas cleaned with a solution of interest contained CT or GC nucleic acids.

### Example 6

### Additional Testing

Another instrument was tested as described in the above examples. Although twelve sites were tested before cleaning, three of the sites were inside the machine and were not cleaned and tested following exposure to the solution of interest.

TABLE 6

| | GC | CT |
|---|---|---|
| # of Areas Swabbed Before | | |
| Instrument Cleaning | | |
| 12 | 9/12 | 3/12 |
| Same Instrument, # of Areas | | |
| Swabbed After Instrument | | |
| Cleaning (3X) | | |
| 9 | 1/9 | 0/9 |
| Same Instrument, # of Areas | | |
| Swabbed After Instrument | | |
| Cleaning | | |
| (1X additional) | | |
| 9 | 0/9 | 0/9 |

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

What is claimed:

1. A solution for removing and helix-destabilizing nucleic acids on surfaces, consisting essentially of surfactant and oxidizing agent, said surfactant and oxidizing agent present in amounts sufficient to render nucleic acids unamplifiable and to permit greater than about 50% of amplification detectable nucleic acids to be removed from said surface.

2. The solution of claim 1, wherein said surfactant is a detergent.

3. The solution of claim 2, wherein said detergent is selected from the group consisting of anionic detergent, non-ionic detergent, ampholytic detergent and zwitterionic detergent.

4. The solution of claim 3, wherein said detergent consists essentially of sodium alkylaryl sulfonate, alcohol sulfate, phosphates and carbonates.

5. The solution of claim 1, wherein the concentration of surfactant is from about 0.5% to about 5%.

6. The solution of claim 5, wherein the concentration of surfactant is from about 0.6% to about 1%.

7. The solution of claim 1, wherein said oxidizing agent is a hypohalous acid or a peroxide.

8. The solution of claim 7, wherein said hypohalous acid is selected from the group consisting of sodium hypochlorite, lithium hypochlorite, calcium hypochlorite and dibasic magnesium hypochlorite.

9. The solution of claim 8, wherein said hypohalous acid is sodium hypochlorite.

10. The solution of claim 1, further comprising a buffer.

11. A kit comprising a first container consisting essentially of a surfactant and a second container consisting essentially of an oxidizing agent

12. The kit of claim 11, wherein said surfactant is a detergent.

13. The kit of claim 12, wherein said detergent is selected from the group consisting of anionic detergent, non-ionic detergent, ampholytic detergent and zwitterionic detergent.

14. The kit of claim 13, wherein said detergent consists essentially of sodium alkylaryl sulfonate, alcohol sulfate, phosphates and carbonates.

15. The kit of claim 11, wherein said oxidizing agent is a hypohalous acid or a peroxide.

16. The kit of claim 15, wherein said hypohalous acid is selected from the group consisting of sodium hypochlorite, lithium hypochlorite, calcium hypochlorite and dibasic magnesium hypochlorite.

17. The kit of claim 16, wherein said hypohalous acid is sodium hypochlorite.

18. A method of decontaminating surfaces previously exposed to amplifiable nucleic acids comprising contacting said surfaces with a solution, wherein said solution consists essentially of surfactant and oxidizing agent, and removing said solution from said surface.

19. The method of claim 18, wherein said surfactant is a detergent.

20. The method of claim 19, wherein said detergent is selected from the group consisting of anionic detergent, non-ionic detergent, ampholytic detergent and zwitterionic detergent.

21. The method of claim 20, wherein said detergent consists essentially of sodium alkylaryl sulfonate, alcohol sulfate, phosphates and carbonates.

22. The method of claim 18, wherein the concentration of surfactant is from about 0.5% to about 5%.

23. The method of claim 22, wherein the concentration of surfactant is from about 0.6% to about 1%.

24. The method of claim 18, wherein said oxidizing agent is a hypohalous acid or a peroxide.

25. The method of claim 24, wherein said hypohalous acid is selected from the group consisting of sodium hypochlorite, lithium hypochlorite, calcium hypochlorite and dibasic magnesium hypochlorite.

26. The method of claim 25, wherein said hypohalous acid is sodium hypochlorite.

27. The method of claim 18, wherein said surfaces are contacted with said solution for at least about 30 seconds.

28. The method of claim 18, wherein said surfaces are contacted with said solution for at least about two minutes.
